(19) United States
(12) Patent Application Publication
(10) Pub. No.: US 2011/0046350 A1
(43) Pub. Date: Feb. 24, 2011

(54) LENGTH-ADJUSTABLE NANOSCALE TETHER FOR BINDING TARGETS TO SUBSTRATES

(75) Inventors: Andrei L. Gindilis, Vancouver, WA (US); Kevin Robert Schwarzkopf, Camas, WA (US)

Correspondence Address: Olympic Patent Works PLLC, P.O. Box 4277, Seattle, WA, 98194-0277, US

(73) Assignee: Sharp Laboratories of America, Inc.

(21) Appl. No.: 12/546,227

(22) Filed: Aug. 24, 2009

(51) Int. Cl. C07K 14/00; C07C 55/02; C07H 21/04

(52) U.S. Cl. 530/350; 562/590; 536/23.1

(57) ABSTRACT

Embodiments of the present invention are directed to reusable, length-adjustable nanoscale tethers that can be incorporated into a variety of different sensors and other electrical, electro-mechanical, electro-optical-mechanical, and optical-mechanical devices. An optionally reusable, length-adjustable nanoscale tether that represents one embodiment of the present invention comprises a binding-structure component, having a substrate-anchor subcomponent and a binding-adapter binding domain, and a binding adaptor that binds to the binding-adapter binding domain and that has a target-binding subcomponent that binds to a target molecule, target particle, or other target entity. The binding-adapter binding domain can be positioned at different distances from the substrate anchor within the binding-structure component so that the distance between a bound target and a sensor substrate can be precisely controlled.

### TECHNICAL FIELD

The present invention is related to sensors, including microscale, sub-microscale, and nanoscale sensors that generate electromagnetic signals when bound to targets and, in particular, to an optionally reusable and length-adjustable nanoscale tether that binds targets to sensor substrates.

### BACKGROUND OF THE INVENTION

Enormous research and development efforts have been made, during the past 100 years, to develop sensors that detect the presence of target molecules, target particles, or other target objects in solutions, air or other gasses, adsorbed to surfaces, or otherwise present in an environment or sample. With the advent of modem microelectronic, sub-microelectronic, microelectromechanical, and sub-microelectromechanical fabrication technologies, a wide variety of different types of sensors have been developed for commercial use. Sensors may be macroscale devices that include arrays of microscale sensor elements, such as oligonucleotide-probe-based microarrays, or may be microscale, sub-microscale, or nanoscale electromechanical, electro-optical, or optical-mechanical subcomponents of microelectromechanical devices, and microfluidic devices. A wide variety of different types of sensors are used in analytical instruments, diagnostics, and scientific instrumentation. As with many other types of technology, sensors are often characterized by various parameters of importance to researchers, designers, and manufacturers of sensor-based devices and equipment, including cost, sensitivity, specificity, viability, reusability, durability, and flexibility in application. Researchers, designers, and manufacturers of sensors and sensor-based devices and equipment continue to seek new sensor technologies that provide low-cost, reliable, durable, reusable, sensitive, and highly specific sensors that can be as broadly applied as possible to a variety of problem domains.

### SUMMARY OF THE INVENTION

Embodiments of the present invention are directed to reusable, length-adjustable nanoscale tethers that can be incorporated into a variety of different sensors and other electrical, electro-mechanical, electro-optical-mechanical, and optical-mechanical devices. An optionally reusable, length-adjustable nanoscale tether that represents one embodiment of the present invention comprises a binding-structure component, having a substrate-anchor subcomponent and a binding-adapter binding domain, and a binding adaptor that binds to the binding-adapter binding domain and that has a target-binding subcomponent that binds to a target molecule, target particle, or other target entity. The binding-adapter binding domain can be positioned at different distances from the substrate anchor within the binding-structure component so that the distance between a bound target and a sensor substrate can be precisely controlled.

### BRIEF DESCRIPTION OF THE DRAWINGS

FIG. 1 illustrates a target-sensing problem domain that provides a context for a description of certain embodiments of the present invention.

FIG. 2 illustrates a sensor-based determination of target concentration within the exemplary problem domain illustrated in FIG. 1.

FIG. 3 illustrates various signal-strength/distance dependencies.

FIG. 4 illustrates additional sensor/target, sensor/non-target, and target/target interactions that may result in a non-linear response of a sensor to target concentration.

FIGS. 5A-E illustrate various relationships between characteristics of a hypothetical sensor.

FIGS. 6A-I describe one class of embodiments of the present invention.

FIG. 7 illustrates a graph of a relationship between the distance from a binding-adapter-binding-domain to a sensor substrate, δ_{y}, and a bound-target-to-substrate distance, δₛ.

FIG. 8 shows alternative types of binding-adaptor components that may be used in alternative embodiments of the nanoscale tether that represent embodiments of the present invention.

FIG. 9 shows symbolic representations of six different states with respect to a sensor substrate and nanoscale tether.

FIG. 10 illustrates three different states of the binding-structure component, binding-adaptor component, and a target/binding-structure/binding-adaptor complex.

FIG. 11 provides a state diagram that illustrates many different possible experimental protocols leading from bare sensor substrates to signal-producing states for sensor/nanoscale-tether/bound-target complexes.

FIG. 12 provides a control-flow diagram for one experimental procedure used with sensors based on the nanoscale tether that represents one embodiment of the present invention.

FIG. 13 illustrates a particular embodiment of the nanoscale tether that represents one embodiment of the present invention.

FIG. 14 illustrates a short oligonucleotide.

FIGS. 15A-B illustrate the hydrogen bonding between the purine and pyrimidine bases of two anti-parallel DNA strands.

FIG. 16 illustrates a short section of a DNA double helix comprising a first strand and a second, anti-parallel strand.

FIG. 17 shows the structural formula for a 5′-monothiolated oligonucleotide.

FIG. 18 shows the structural formula for biotin.

FIG. 19 shows the structural formula for succinimidyl 4-[N-maleimidomethyl]cyclohexane-1-carboxylate (“SMCC”).

### DETAILED DESCRIPTION OF THE INVENTION

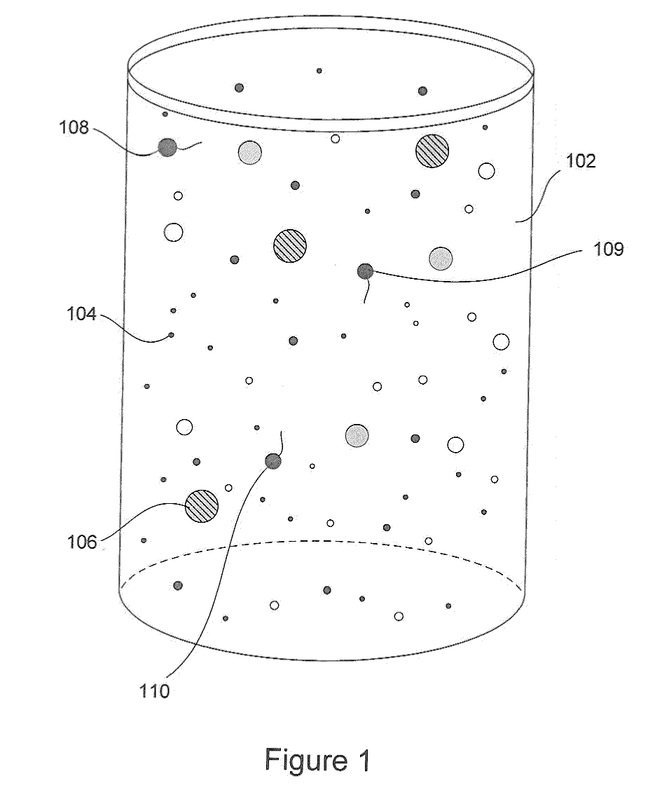

FIG. 1 illustrates a target-sensing problem domain that provides a context for a description of certain embodiments of the present invention. FIG. 1 shows a small, enclosed volume 102 of a complex solution containing symbolic representations of various different small molecules and ions, such as small molecule 104, various non-target biopolymers, such as non-target biopolymer 106, as well as a target biopolymer, three instances of which 108-110 are shown in the small enclosed volume 102 illustrated in FIG. 1. FIG. 1 is not intended to provide an accurate, scale-correct rendering of an enclosed volume of a complex solution, but is instead intended to illustrate the complexity of the solution 102 as well as to introduce symbolic illustration conventions for sensor targets and for non-target entities within the solution. Considering the enclosed volume of solution to be a sample, one common problem domain constitutes determining the concentration of target entities 108-110, [t], in the small volume 102 of the solution illustrated in FIG. 1.

There are many highly accurate, mature, and generally complex technologies for determining concentrations of targets in solutions and other media. These methods include a wide variety of different types of chromatography techniques, gel electrophoresis, analytical centrifugation, fluorescent-antibody assays, and many other methods. In general, any particular method, particularly the classical biochemical methods, can be used only for a subset of the sensing problem domains. For example, many methods require a minimum volume of solution for analysis. In addition, methods generally can detect targets reliably only over particular concentration ranges. Targets may often interact with each other and/or with other molecules, particles, or other entities in the solution in ways that interfere with accurate determination of target concentration. Many methods require particular solvents, and cannot be used for other solvents. Many methods are time-consuming, expensive, and require complex instrumentation and laboratory equipment, and cannot therefore be carried out in real time or under field conditions.

For all of the above reasons, enormous effort has been undertaken, in recent years, to develop and commercialize highly specific, inexpensive, small, reliable, and sensitive sensors for detecting a wide variety of different targets in solutions, in air, adsorbed to different surfaces, substrates, and entities, and in other media and environments. The sensors may be used for environmental monitoring, biowarfare-agent detection, explosives detection, analysis of biopolymer and small-molecule solutions, detection of impurities in manufacturing quality control, and for a wide variety of additional applications, including diagnostics and scientific-research applications.

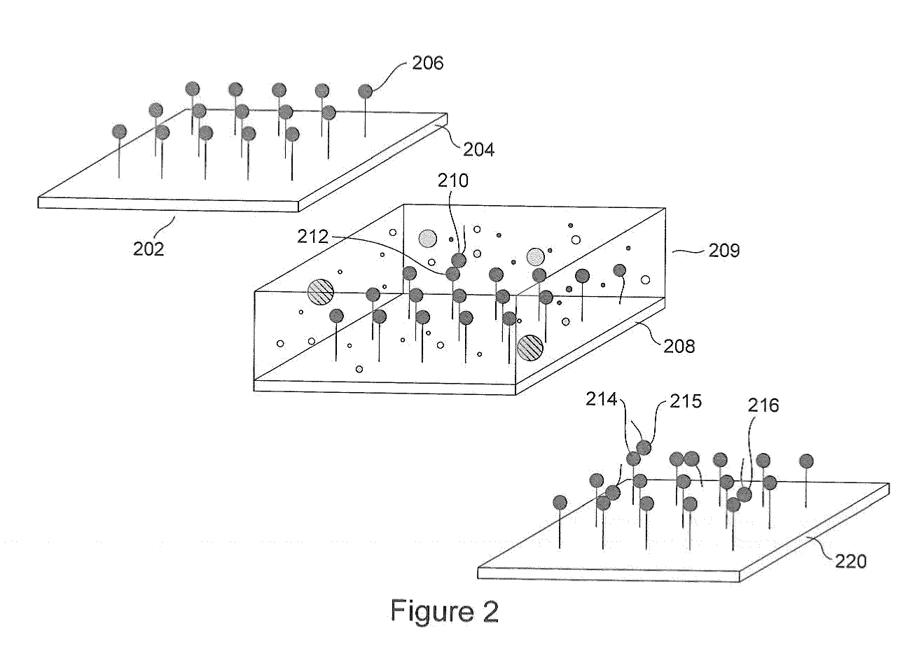

In general, a sensor is a signal transducer that responds to a target concentration or target presence, in a defined environment, by generating an electromagnetic signal, including electrical current and voltage signals, optical signals, radio-frequency signals, and other types of signals that can be detected and quantitatively evaluated by electronic devices, generally microelectronic, microprocessor-controlled devices. FIG. 2 illustrates a sensor-based determination of target concentration within the exemplary problem domain illustrated in FIG. 1. In FIG. 2, a small portion 202 of a sensor is shown to include a substrate 204 and target-binding probes, such as probe 206. Different types of sensors may include different numbers of probes, from one to millions, hundreds of millions, or billions of probes that may be organized into arrays of other structures. In the following discussion, a single-probe-type sensor is discussed. For example, in the portion of the sensor 202 shown in FIG. 2, all of the probes, such as probe 206, are designed to bind, or tightly associate with, one particular type of target. However, sensors may include many different types of probes, multi-functional probes that bind to, or associate with, multiple types of targets, and various different types of chemical and/or electromechanical probes, and embodiments of the present invention are applicable to all of these different types of sensors.

In other to determine the target concentration in the exemplary solution shown in FIG. 1, the sensor is exposed to a small volume of solution 209, shown above the sensor substrate 208 in FIG. 2. Targets within the solution, such as target 210, randomly collide with, and bind to, probes, such as probe 212. Binding may occur by various different types of binding interactions, including ionic interactions, hydrogen bonding, Van der Waals interactions, covalent bonding, electrostatic surface attractions, and other types of binding. In many cases, binding interactions are quite specific. Targets may bind to probes with binding constants of many orders of magnitude greater than non-targets, including specific binding of biopolymer probes containing binding sites for specific small-molecule or biopolymer targets. Enzymes bind to small-molecule substrates, antibodies bind to specific antigens, and DNA-binding proteins and RNA biopolymers may bind with high specificity to particular monomer subsequences. However, useful sensors may also include less specific probes that bind less specifically to an entire class of targets.

In certain cases, after an adequate exposure time, the sample solution is washed away from the sensor surface, leaving targets bound to a certain percentage of the probes, including bound targets 214-216 in FIG. 2. In other cases, including sensors used for certain types of impedance spectroscopy, a washing or solution-substitution step in not needed. The percentage of probes bound to targets is generally representative of the concentration of target in the sample solution. Following a washing or solution-substitution step, in certain cases, or during the binding process, in other cases, the sensor is then queried, by any of various electrical or optical means, in order to detect a signal indicative of the density of bound targets on the sensor substrate, in turn indicative of target concentration in the sample solution. For example, chemiluminescent-compound-labeled biopolymer targets bound to oligonucleotide probes of a microarray can be detected by illuminating the microarray with light of a first wavelength and detecting light of a second, generally longer wavelength emitted by the chemiluminescent labels. In another example, the surface plasmon resonance technique detects biopolymers adsorbed to surfaces by plasmon-induced changes in light reflected from the surface, including changes in reflection angles, wavelengths, and other changes in the reflected light. In microcantilever or nanocantilever transducers, mechanical bending of the nanocantilever as a result of the weight of adsorbed molecules generates a current or voltage signal that varies with the degree to which the cantilever is bent. Atomic-force microscopes move a tiny, needle across a surface, positioned at a constant distance from the surface by feedback control, which generates electrical signals as the needle rises and falls as it passes over substrate atoms and adsorbed ions and molecules. As yet another example, fluorescence-resonance-energy-transfer-based detectors may detect the proximity of different fluorescent labels bound to targets and probes by emitting a light signal of a particular wavelength only when targets are bound to, or tightly associated with, probes so that the two different fluorophores are within some maximum distance from one another. Finally, in sensors used for impedance spectroscopy, the target-to-probe binding process is detected, while it occurs and with no washing step, by detecting a change in the impedance of an electrode at a frequency at which impedance changes are proportional to bound target density, in turn proportional to target concentration. Impedance spectroscopy takes advantage of the fact that binding of target to probe changes the capacity of the electrode surface.

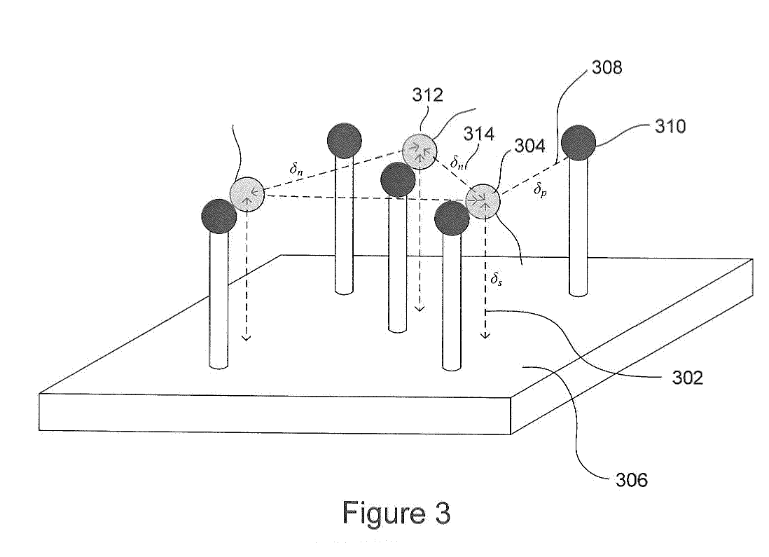

In many cases, the signal generated by targets bound to a probe is strongly dependent on various distance and geometrical considerations. FIG. 3 illustrates various signal-strength/distance dependencies. For example, as shown in FIG. 3, the intensity or strength of the generated signal may strongly depend on the distance δₛ 302 between bound targets, such as bound target 304, and the surface 306 of the sensor substrate. A good example of target-to-surface signal intensity dependence occurs both in certain types of fluorescent-resonance-energy-transfer-based sensors and in surface-plasmon-resonance-based sensors. Signal strength may also depend on the minimum distance δₚ 308 between a bound target 304 and a neighboring probe 310 and the minimum distance between a bound probe 304 and a neighboring bound probe δₙ 314. As one example, light emitted by a first bound target 304 may be partially absorbed by a neighboring bound target 312, so that, as the density of targets adsorbed to the sensor surface increases, the aggregate signal intensity of the bound targets is dampened, leading to a non-linear sensor response to incident light.

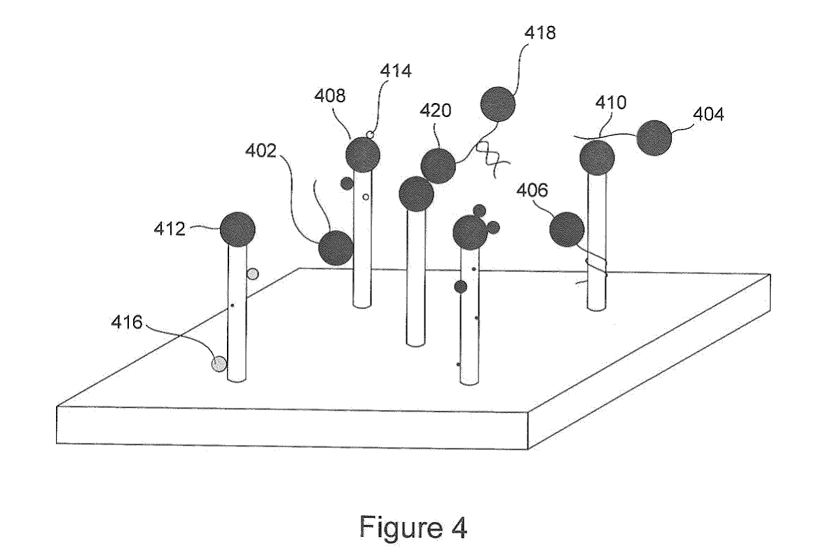

FIG. 4 illustrates additional sensor/target, sensor/non-target, and target/target interactions that may result in a non-linear response of a sensor to target concentration. In FIG. 4, various targets, including target 402, 404, and 406 have bound nonspecifically to probes 408 and 410. The probes are designed so that a target-binding feature of the probe, represented in FIGS. 2-4 by a darkened disk-shaped feature, such as feature 412, bind with high specificity to targets. In FIG. 3, for example, targets 304 and 312 are bound specifically, as intended, to their respective probes. However, targets may nonspecifically bind to probes, either by binding or associating with non-binding-domain portions of the probe, such as targets 402 and 406 in FIG. 4, or by the association of a non-target-binding-domain region of the target to the binding domain of the probe, such as target 404 in FIG. 4. In addition, non-target molecules or particles, such as non-target molecules 414 and 416, may non-specifically bind to probes, and targets, such as target 418, may bind to, or associate with, other, bound targets 420. In general, non-specific binding of targets to sensor probes and non-specific binding of non-target entities to sensor probes may result in highly non-linear sensor-response curves, signal responses that do not reflect the target density of bound targets, and small signal-to-noise ratios.

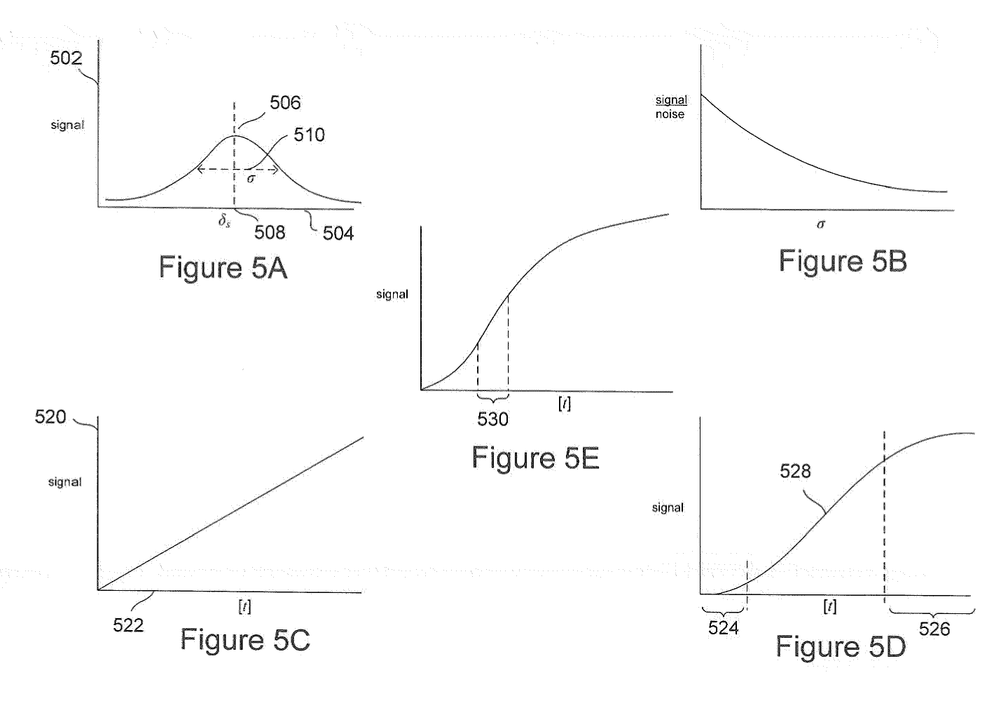

FIGS. 5A-E illustrate various relationships between characteristics of a hypothetical sensor. In FIG. 5A, the relationship between signal intensity and target-to-substrate distance is plotted for a hypothetical sensor. The signal intensity is plotted with respect to a vertical axis 502 and the target-to-substrate distance, δ_{s,} is plotted with respect to a horizontal axis 504. As illustrated in FIG. 5A, the relationship between signal intensity and bound-target-to-substrate-distance δₛ has the form of a Gaussian curve, with the signal intensity greatest 506 at an optimal bound-target-to-substrate distance 508. In order to achieve best sensor response, it would be desirable to design sensor probes so that specifically bound targets reside at the optimal bound-target-to-substrate distance 508.

A characteristic of curves, such as that shown in FIG. 5A, is the breadth of the curve, σ 510, often measured as the width of the curve at ½ peak height. In the case that σ is relatively small, and the curve correspondingly narrow, the contribution of specifically bound targets to the aggregate signal generated by all targets bound to the sensor, when specifically bound targets are positioned at the optimal bound-target-to-substrate distance, will be generally far greater than the undesired signal produced by non-specifically bound targets, such as non-specifically bound targets 402 and 406 in FIG. 4, since the non-specifically bound targets are generally located at distances from the sensor substrate less than the optimal bound-target-to-substrate distance. Thus, as shown in FIG. 5B, the signal-to-noise ratio may strongly depend on σ, or the breadth of the curve shown in FIG. 5A. Small values of σ lead to large signal-to-noise ratios.

FIG. 5C shows a linear sensor-response curve, in which the signal intensity, plotted with respect to vertical axis 520, is a linear function of target concentration [t], plotted with respect to the horizontal axis 522. Linear sensor-response curves are desirable. Sensors exhibiting linear responses are easily calibrated and provide accurate estimates of target concentrations over a wide range of target concentrations. In many problem domains, linear sensor-response curves, such as that shown in FIG. 5C, are not observed, for a variety of reasons, including non-specific binding of target to the sensor as well as non-specific binding of non-target entities to the sensor, as discussed above with reference to FIG. 4. In general, the response curves for sensors tend to be non-linear, and sigmoidal in shape. FIG. 5D shows a desirable sensor-response curve in which non-linearities occur at very low target concentrations 524 and at relatively high target concentrations 526, with an essentially linear response curve 528 across a broad range of target concentrations. As non-specific binding of target and non-target entities to sensors increases in proportion to specific-binding of target to sensors, as shown in FIG. 5E, the useful linear portion of a response curve 530 may cover only a very small range of target concentrations. In worst cases, a response curve is entirely non-linear. Calibration of sensors that exhibit linear response curves, such as the hypothetical response curve shown in FIG. 5C, can be accomplished by adjusting a single, intercept parameter or, at worst, an intercept parameter and a slope parameter. By contrast, non-linear response curves may require complex curve-fitting calibration and, in many cases, exhibit relatively large changes and perturbations as a result of even relatively small changes in sensing conditions and sensor characteristics.

Thus, as discussed above, the sensitivity, signal-to-noise ratio, and accuracy of a sensor may depend significantly on the precision at which a bound target is maintained at an optimal distance from the sensor surface and/or the minimal distance between different bound targets. Furthermore, sensor probes need to exhibit high specificity for targets and provide as few opportunities for non-specific binding by both targets and non-target entities as possible, in order to ensure accurate sensor response to target concentration and high signal-to-noise ratios. Certain types of sensors are designed to detect the presence of a target, rather than to quantify target concentration over a range of possible concentrations. Such sensors generally exhibit highly non-linear, threshold-concentration-based responses, in the best case a binary response, with little or no signal produced when the target concentration falls below a threshold concentration and a maximum signal produced when the target concentration falls above the threshold concentration. For such binary sensors, ensuring that bound-target-to-substrate distances fall within a narrow range of distances, and that a minimum distance between bound targets is greater than a threshold value, may also be important to ensure high signal-to-noise ratios and an accurate threshold-concentration response.

Reusability and flexibility of application are additional, important characteristics for commercial, sensor-based devices and equipment. Many currently available microscale, sub-microscale, and nanoscale sensors are essentially single-use sensors or single-application sensors. In many cases, once the sensor has been exposed to a target-containing medium, the sensor probes cannot be reliably restored to their pre-target-exposure states. For example, targets may bind with sufficient strength to sensor probes that the targets cannot be subsequently removed without also removing, or deleteriously affecting, the probes. Even in the case that the sensor may be reused, it is often the case that the probes are extremely application specific, designed specifically for a particular target entity and a particular analytical method. Although certain types of non-reusable and application-specific sensors may be mass-produced cheaply, replacement and recalibration of replaced, non-reusable and/or application-specific sensors may, in certain cases, represent significant cost and inconvenience to those using the non-reusable and application-specific sensors for a variety of sensing tasks. For all of these reasons, designers, manufacturers, and consumers of microscale sensors, sub-microscale sensors, and nanoscale sensors continue to seek new and improved sensor technologies that provide high specificity, accuracy, high signal-to-noise levels, and linear response curves as well as providing, in certain cases, reusability of all or a portion of the sensors as well as flexibility in application.

Embodiments of the present invention are directed to length-adjustable nanoscale tethers that are anchored to a sensor substrate or surface and that specifically bind to targets. In certain cases, the length-adjustable tethers comprise multiple components, one or more of which are common for many applications and, in many cases, reusable. By replacing an adapter component in the multi-component tether, the tether can be flexibly adapted for binding different types of classes of targets and for different types of analytical techniques with different optimal bound-target-to-substrate distances. The tethers can be adjusted to maintain a precise bound-target-to-substrate distance, and can be reused and reconfigured to provide both reusability of sensors and flexibility in application of sensors to multiple targets and target-containing media. It should be noted that, even when a sensor based on the length-adjustable nanoscale tethers that represent embodiments of the present invention are not reusable, the ability to precisely control bound-target-to-substrate distances by using length-adjustable nanoscale tethers provides great advantages in increasing signal-to-noise ratios and increasing the breadth of linear response portions of a sensor response curve.

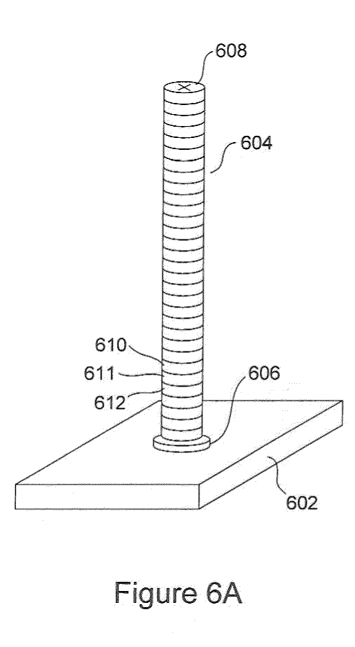

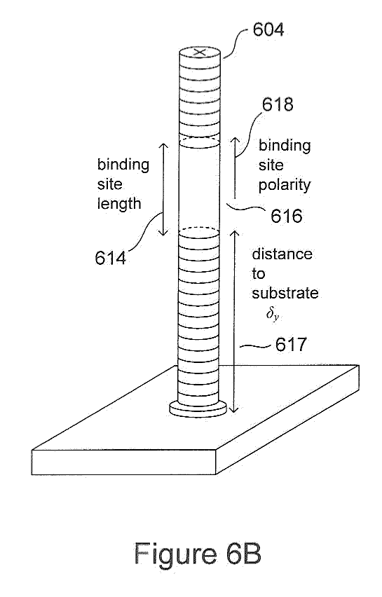

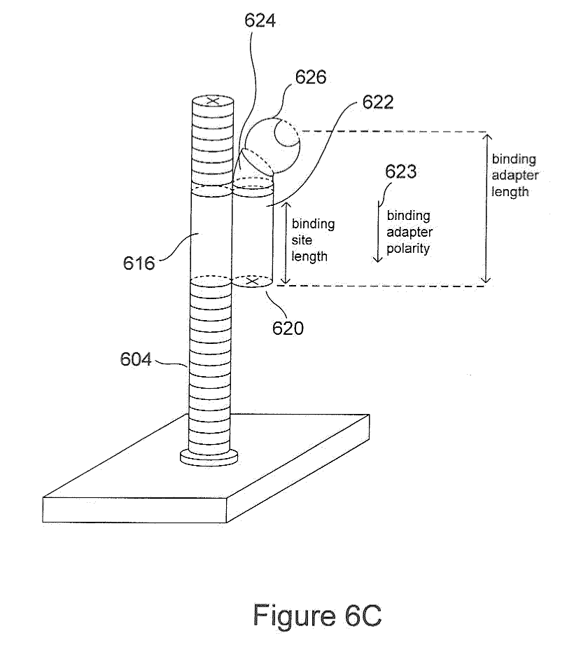

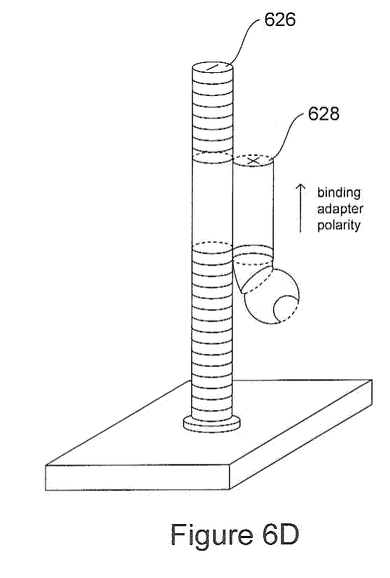

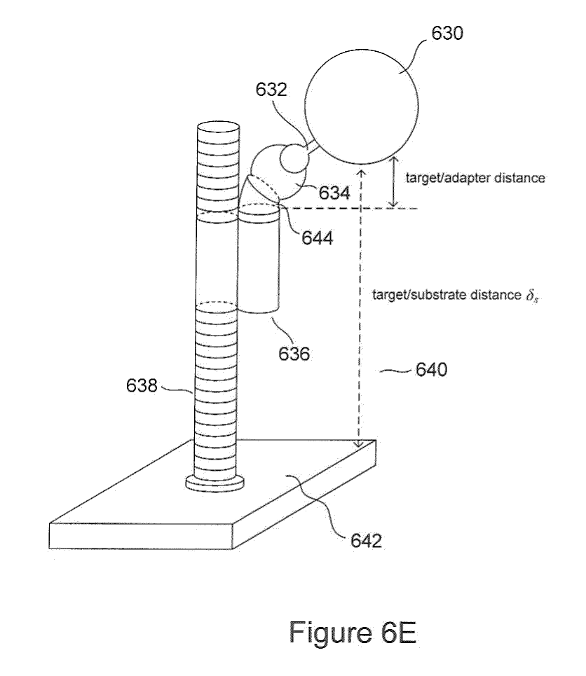

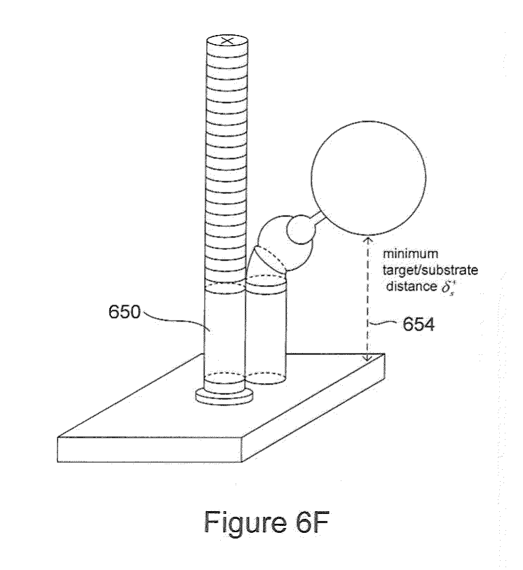

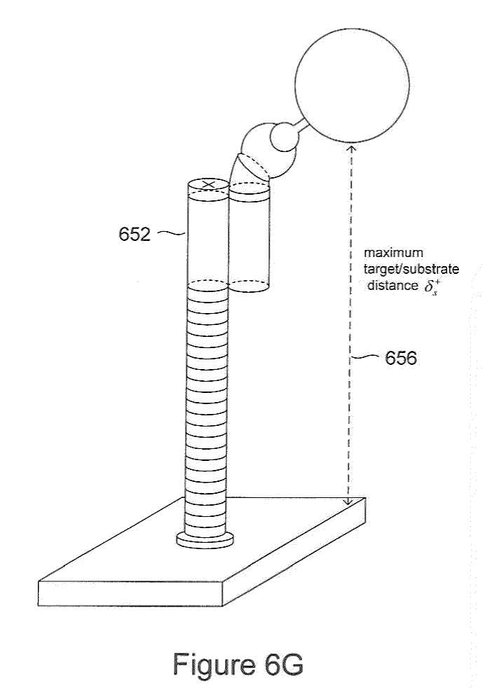

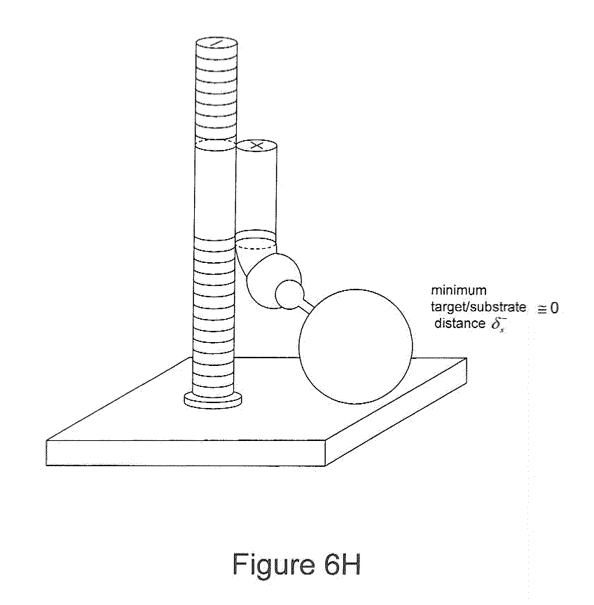

FIGS. 6A-I describe one class of embodiments of the present invention. All of these figures use the same illustration conventions, introduced over the sequence of FIGS. 6A-E. FIG. 6A shows a portion of a sensor surface, or substrate, to which a binding-structure component of a nanoscale tether that represents one embodiment of the present invention is anchored. In FIG. 6A, and in the remaining FIGS. 6B-I, the portion of the sensor substrate 602 is shown as a planar, rectangular layer. The binding-structure component 604 is anchored to the sensor substrate by a substrate anchor 606. The binding-structure component 604, in certain embodiments of the present invention, has a polarity with respect to the axis of the binding-structure component that is approximately normal to the substrate. In FIG. 6A, the polarity is indicated by the “+” label 608 on the end of the binding-structure component furthest from the sensor substrate, with an implied label “−” at the opposite of the binding-structure component. Finally, the binding-structure component 604 comprises regularly spaced and physically differentiable segments, such as segments 610-612. A contiguous subset of these segments, as discussed further below, comprises a binding domain to which a binding-adaptor component of the tether is affixed.

FIG. 6B illustrates a binding domain of a binding-structure component of a nanoscale tether that represents one embodiment of the present invention. In the example shown in FIG. 6B, a binding-adaptor-component binding site 616, of a length indicated by double-headed arrow 614, comprises seven contiguous segments of the binding-structure component at a distance δ_{y} 617 from the substrate, where, in FIG. 6B, δ_{y} is equal to the length of 16 segments. The binding site 616 has a binding-site polarity 618 equivalent to the polarity of the binding-structure component 604.

FIG. 6C illustrates a binding-adaptor component affixed to the binding-structure component of a nanoscale tether that represents one embodiment of the present invention. As shown in FIG. 6C, the binding-adaptor component 620 includes a segmented region 622 complementary to the binding site 616 of the binding-structure component 604, but with opposite polarity 623 with respect to the polarity of the binding-structure component 604. In addition, the binding-adaptor component 620 includes a linker region 624 that attaches a target-binding component 626 to the segmented-binding domain 622.

FIG. 6D shows an alternative embodiment of the nanoscale tether, discussed above with reference to FIGS. 6A-C, in which the polarities of the binding-structure component and the binding-adaptor component are reversed. Thus, in FIG. 6D, the far end of the binding-structure component 626 is labeled with a “−” label while the complementary end of the binding-adaptor component 628 is labeled with a “+” label. In the embodiments discussed with reference to FIGS. 6A-I, the binding-structure and binding-adaptor-component polarities are opposite from one another.

FIG. 6E shows a nanoscale tether, which represents one embodiment of the present invention, to which a target is bound. In FIG. 6E, the target 630 includes a binding domain 632 complementary to the target-binding subcomponent 634 of the binding-adaptor component 636 affixed to the binding-structure component 638 of the nanoscale tether. As shown in FIG. 6E, the target 630 is held at a particular target/substrate distance δₛ 640 from the substrate surface 642. In certain cases, the target-binding domain 632, binding-adaptor binding component 634, and binding-adaptor link component 644 may be somewhat flexible, so that the target/substrate distance δₛ may vary over a range of min(δₛ) to max(δₛ). In general, the range min(δₛ)-to-max(δₛ) is relatively short with respect to the average bound-target-to-substrate δₛ.

As shown in FIGS. 6F-G, with the binding-structure component oriented with the end labeled “+” away from the substrate, the binding domain of the binding-structure component can be positioned anywhere along the length of the binding-substrate component from a minimal-δₛ position 650 to a maximum δₛ position 652 in FIG. 6G. Thus, the position of the binding-adaptor component with respect to the binding-structure component can be fixed at intervals equal to the length of a binding-structure-component segment over the range of positions from the minimal δₛ position 650 in FIG. 6F to the maximum δₛ position 652 in FIG. 6G. Using a convention that the polarity of the nanoscale tether is the same as the polarity of the binding-structure component, distances 654 and 656 in FIG. 6F and 6G are referred to as δₛ⁺, with distance 654 in FIG. 6F equal to min(δₛ⁺)and distance 656 in FIG. 6G equal to max(δₛ⁺). FIGS. 6H-I illustrate the nanoscale tether that represents one embodiment of the present invention with a polarity opposite from that shown in FIGS. 6F-G with respect to the sensor substrate. In the opposite-polarity case, the minimum target/substrate distance min(δₛ⁻) is the closest-approach distance of the substrate and target, and the maximum target/substrate distance max(δₛ⁻) 660 is relatively small in comparison to max(δₛ⁺) (656 in FIG. 6G). The relative ranges of δₛ depend on the relative sizes and positions of the bound target and the binding-adaptor component. In the case that the bound target cannot approach the substrate closer than the binding-adaptor component, the range of δₛ obtained by varying the position of the binding-adaptor binding domain on the binding-structure component may be equivalent for either polarity of the nanoscale tether.

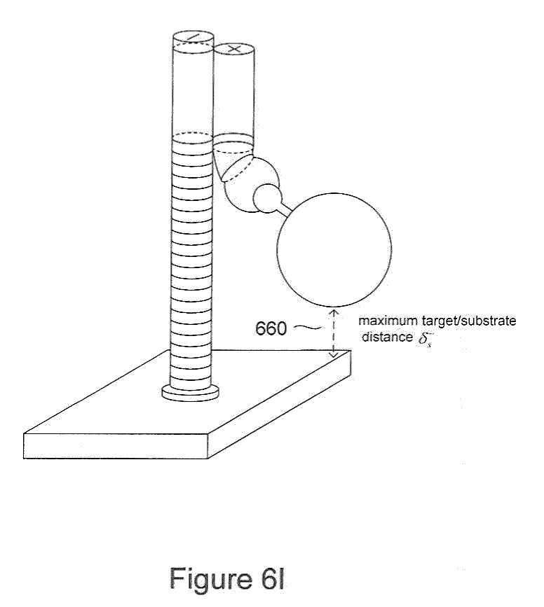

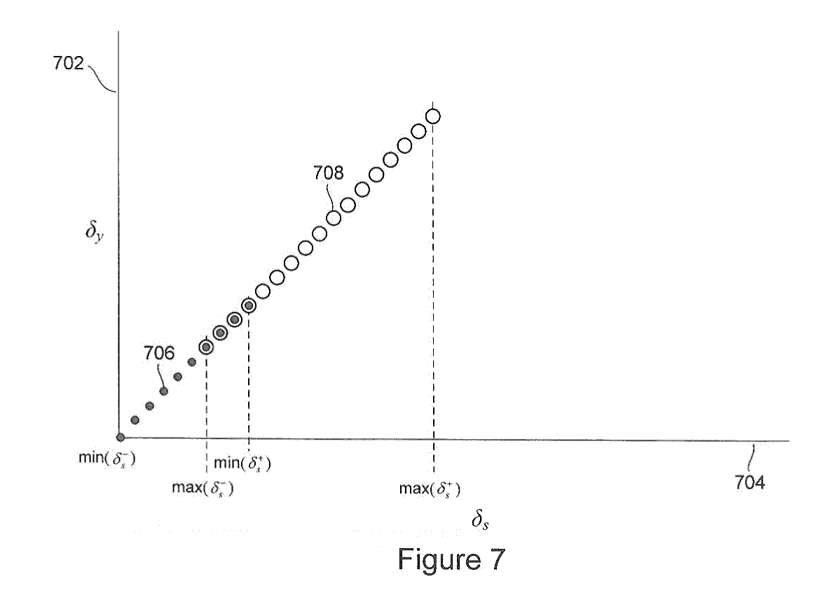

FIG. 7 illustrates a graph of a relationship between the distance from a binding-adapter-binding-domain to a sensor substrate, δ_{y}, and a bound-target-to-substrate distance, δₛ. The filled circles, such as filled circle 706, represent binding positions for the tether orientation discussed with reference to FIGS. 6H-I, and the open circles, such as open circle 708, refer to positions available for the nanoscale tether in the orientation discussed with reference to FIGS. 6F-G. The distances min(δₛ⁻), max(δₛ⁻), min(δₛ⁺), and max(δₛ⁺), discussed above with reference to FIGS. 6F-I, are plotted along the horizontal axis 704. As illustrated in FIGS. 6G-I and FIG. 7, the nanoscale tether that represents one embodiment of the present invention can be adapted, by placing the binding-adaptor binding site in different positions along the binding-structure component and by changing the nanoscale-tether polarity, to hold a bound target at any of a large number of discrete positions regularly spaced within the range min(δₛ⁻) to max(δₛ⁺). Thus, the nanoscale tether that represents one embodiment of the present invention is length-adjustable. As discussed above, the length-adjustable nature of the nanoscale tether provides for wide linear-response ranges and high signal-to-noise ratios.

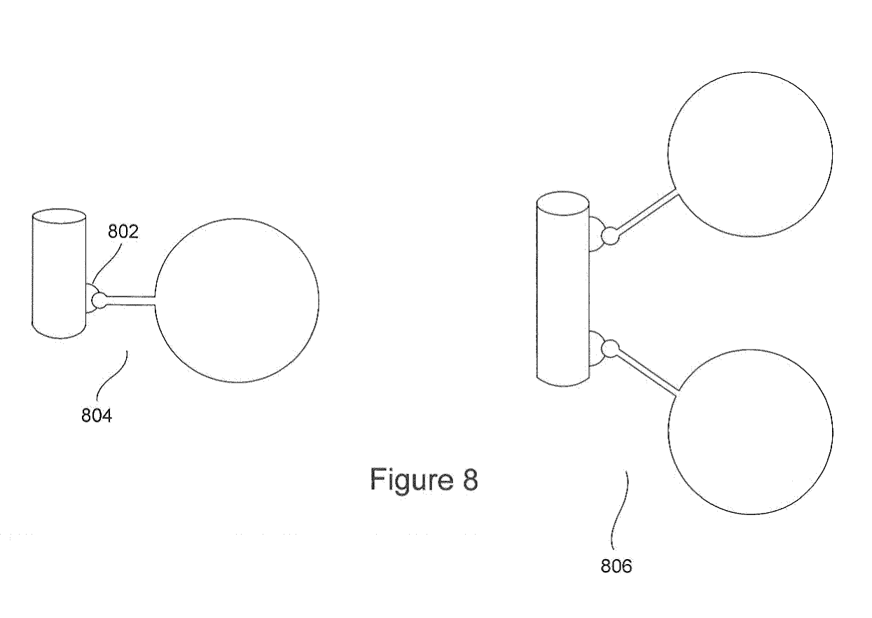

FIG. 8 shows alternative types of binding-adaptor components that may be used in alternative embodiments of the nanoscale tether that represent embodiments of the present invention. The binding component of the binding-adaptor component 802 may be affixed to a position within the binding domain, rather than linked to ends of the binding domain, as shown in binding-adaptor-component/target complex 804 in FIG. 8. In certain embodiments, the binding-adaptor component may be multivalent, as shown in binding-adaptor/target complex 806 in FIG. 8. A wide variety of different types of binding-adaptor components and binding-structure components are available.

The combination of the binding-structure and binding-adapter components greatly enhances flexible application and reusability of sensors that include nanoscale tethers of the present invention. First, as discussed above, nanoscale tethers can be flexibly reconfigured for different targets by changing binding-adapter components, rather than replacing the entire nanoscale tether. In many embodiments of the present invention, the binding-adapter-component binding domain of the binding-structure component is defined by complementarity of the binding domain within the binding-adapter component, so that a bound-target-to-substrate distance can be altered by changing the binding-adapter component. In general, the ability to reuse binding-structure components anchored to a sensor substrate can provide savings in time and money by eliminating the need to fabricate a completely new sensor for each application and experiment.

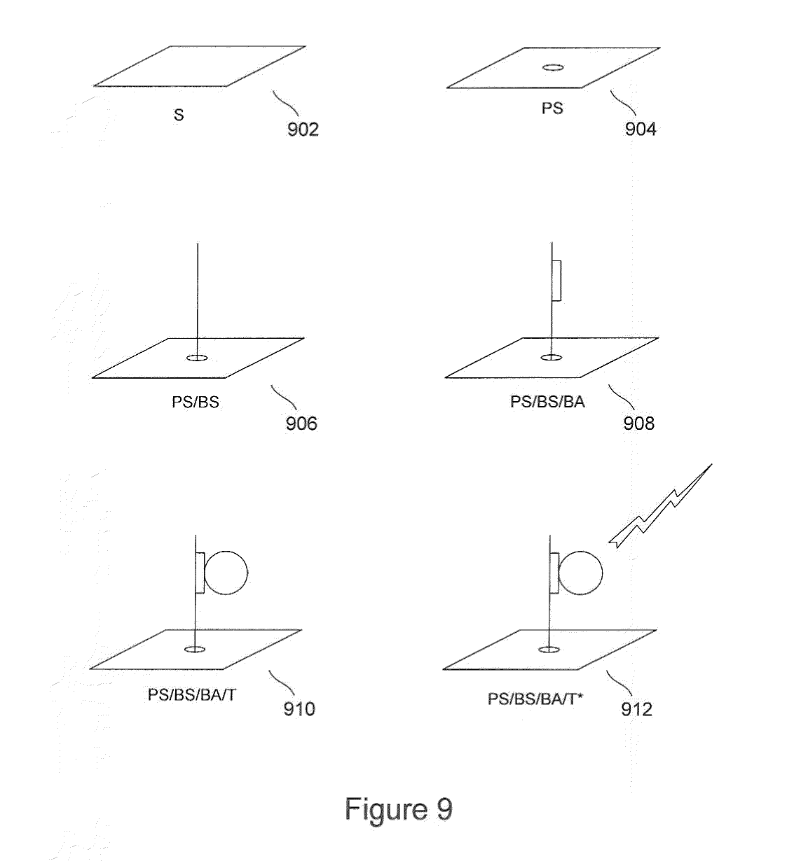

FIG. 9 shows symbolic representations of six different states with respect to a sensor substrate and nanoscale tether. A sensor substrate may be in an initial, unprepared state 902. The initial substrate may be prepared, by any of many different substrate-preparation methods, to produce a prepared substrate 904 to which a binding-structure component of the nanoscale tether can be bound. The initial substrate state is referred to as “S,” and the prepared substrate state is referred to as “PS.” The binding structure can be bound, by a generally reversible process, to a prepared substrate to produce the “PS/BS” state 906. The binding adaptor can be bound to the binding substrate to produce the “PS/BS/BA” state 908. A target can be bound, during a sensing step, to the binding-adaptor, component to produce the state “PS/BS/BA/T” 910. Finally, a sensor containing bound targets can be queried, by any of various different optical or electromagnetic inputs, to generate a signal, the signal-generating state referred to as “PS/BS/BA/T*” 912.

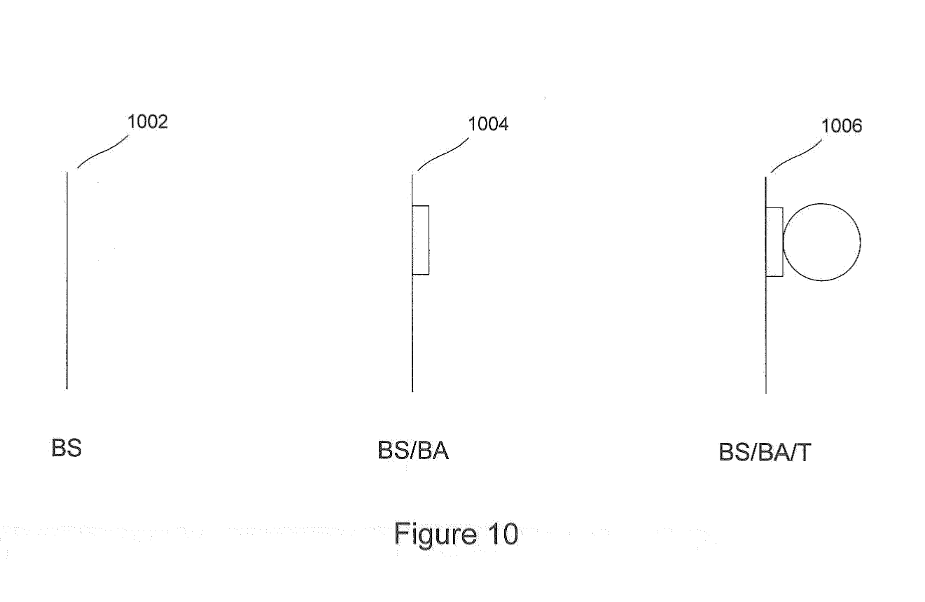

FIG. 10 illustrates three different states of the binding-structure component, binding-adaptor component, and a target/binding-structure/binding-adaptor complex. The three states include the “BS” state 1002, an unbound binding-structure component, the “BS/BA” state 1004, a binding-structure component to which a binding-adaptors is bound, and the “BS/BA/T” state 1006, in which a target is bound to the binding-adaptor component, in turn bound to the binding-structure component.

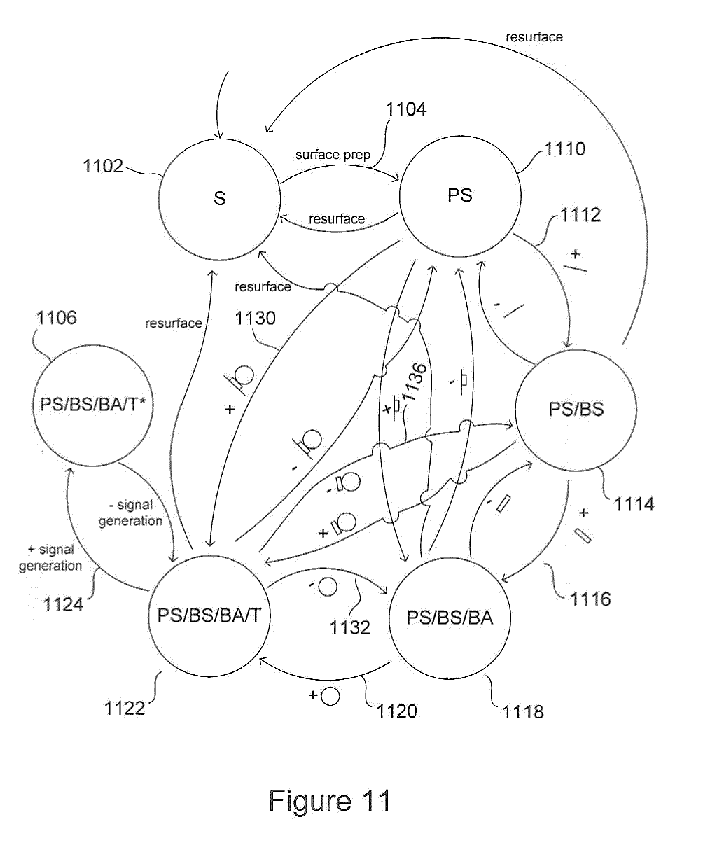

The illustration conventions discussed above with respect to FIGS. 9 and 10 are used, in a next figure that provides a state diagram for various different transitions between sensor substrate and nanoscale-tether states. FIG. 11 provides a state diagram that illustrates many different possible experimental protocols leading from bare sensor substrates to signal-producing states for sensor/nanoscale-tether/bound-target complexes. In FIG. 11, circles, such as circle 1102, represent the various different states illustrated in FIG. 9. Curved arrows, such as curved arrow 1004, represent state transitions. A single sensing operation comprises a path from the state “S” 1102 to the signal-producing state “PS/BS/BA/T*” 1106 by any allowed path comprising one or more transitions and intervening nodes. For example, an unprepared sensor substrate, in state 1102, can be prepared, according to transition 1104, to produce a prepared substrate represented by state 1110. The prepared substrate can then be exposed to binding-structure components, in transition 1112, to bind binding-structure components to the prepared substrate in order to produce state “PS/BS” 1114. The binding-adaptor components can be added, via transition 1116, to produce state “PS/BS/BA” 1118 which, when exposed to targets, via transition 1120, produces state “PS/BS/BA/T” 1122. Finally, exposing the sensor with bound target to a query signal or other signal-generation-inducing event, via transition 1124, produces the signal-generation state 1106. However, in an alternative experimental protocol, the target may be bound to a free binding-structure/binding-adaptor complex, in solution, via transition 1130, to directly produce state “PS/BS/BA/T” 1122 from state “PS” 1110. In this experimental protocol, the signal-generating state 1106 can be reached without traversal of states 1114 and 1118. Note that most transitions between nodes are reversible. In the case that removing target from binding-adaptor component can be carried out without disturbing the binding-structure/binding-adaptor complex, via transition 1132, then following signal generation, the bound target can be removed, returning to state “PS/BS/BA” from which another sensing cycle, including exposure of the sensor to targets, can be carried out without re-traversing states 1102, 1110, and 1114. For any particular sensor, probe, and target combination, only certain of the state transitions shown in FIG. 11 may be feasibly included in a sequential experimental protocol. Nonetheless, in general, the nanoscale tethers that represent embodiments of the present invention can allow for many different types of experimental protocols and degrees of usability and reusability.

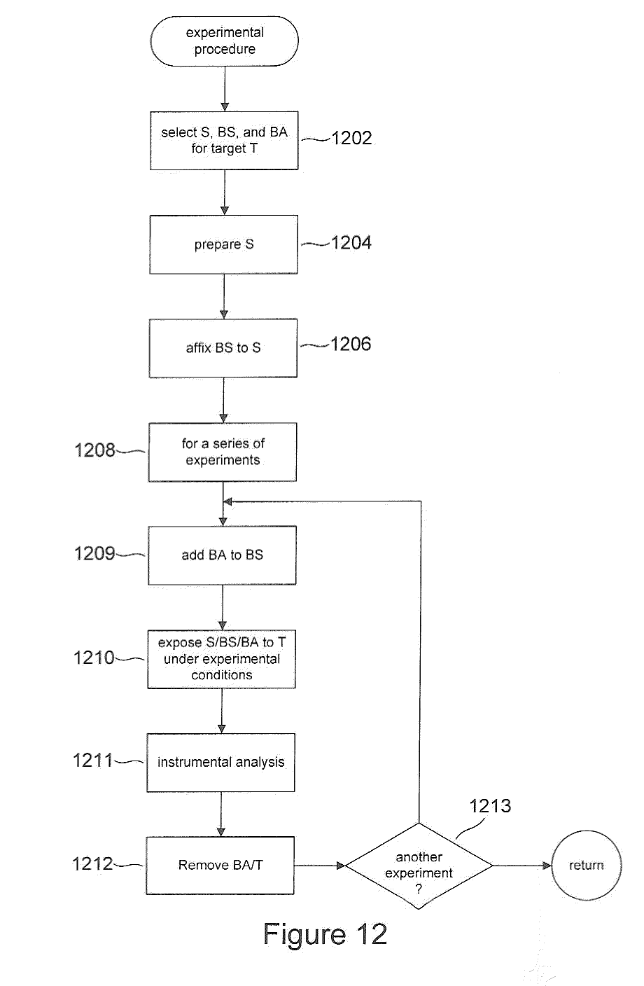

FIG. 12 provides a control-flow diagram for one experimental procedure used with sensors based on the nanoscale tether that represents one embodiment of the present invention. In step 1202, a sensor substrate, binding-structure component, and binding-adaptor component are selected for a particular target that is to be sensed by the sensor. Selection of the binding-structure component, for example, includes determination of the distance between the binding-adaptor binding domain and the substrate, δ_{y}, that in turn determines the bound-target-to-substrate distance δₛ. Next, in step 1204, the substrate is prepared to enable binding-structure components to be bound to the substrate. Substrate preparation may include steps to ensure a threshold maximum bound-target-to-bound-target distance, to improve signal-to-noise ratios and to optimize sensor-response curves in order to achieve broad linear response ranges. For example, a nanoscale array of anchors or binding components may be fabricated on the surface of the sensor by chemical, nanoscale imprint lithography, self-assembly, or by other means. Next, in step 1206, the binding-structure components are fixed to the sensor substrate by any of various different methods, including covalent binding, ionic binding, nanoscale chemical-mechanical recognition and binding, and by other means. Then, a series of different experiments may be performed in the for-loop of steps 1208-1213. In each experiment, binding-adaptor components are affixed to binding-structure components, in step 1209, the sensor is exposed to target, in step 1210, and analysis is carried out, in step 1211, in order to record a signal generated by the target bound to the sensor. Then, in step 1212, the binding-adaptor-component/target complex is removed from the binding-structure component, via transition 1136 in FIG. 11, and, when another experiment is desired, as determined in step 1213, control flows back to step 1209. Again, as discussed above with reference to FIG. 11, there are many hundreds of different possible experimental procedures that can be implemented using the nanoscale tether that represents embodiments of the present invention, depending on the nanoscale-tether components, targets, and degree of reversibility of the various transitions illustrated in FIG. 11.

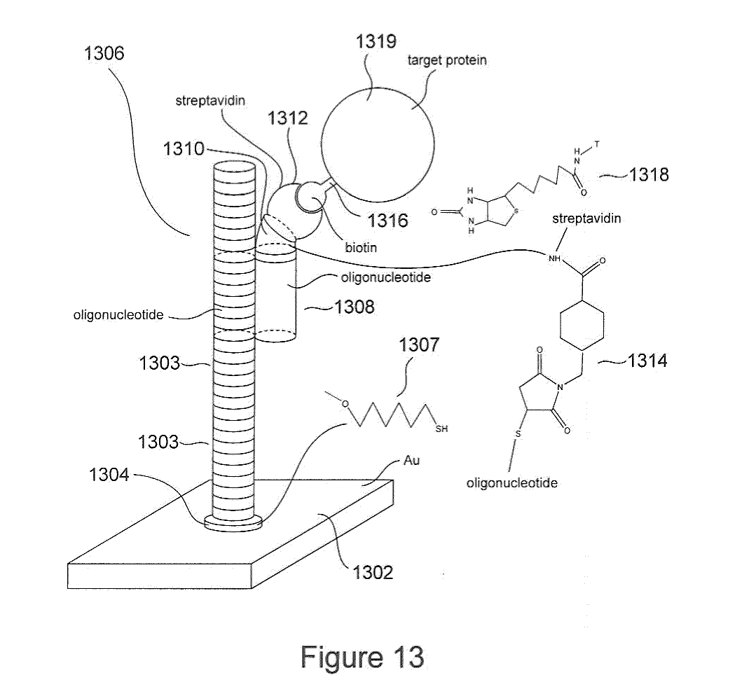

FIG. 13 illustrates a particular embodiment of the nanoscale tether that represents one embodiment of the present invention. In this embodiment, the surface of the sensor substrate 1302 is a thin gold film. The linking component 1304 that fastens the binding-structure component 1306 to the substrate 1302 is a thiol-functionalized alcohol group 1306 bound to a phosphodiester phosphorous atom at the 5' end of an oligonucleotide. The binding-structure complex is an oligonucleotide, with nucleotide segments 1306. The binding domain of the binding-adaptor component is also an oligonucleotide, with opposite polarity from the binding-structure oligonucleotide 1308. The binding-adaptor component recognizes and binds to the binding domain of the binding-structure component through complementary Watson-Crick binding. The linker component 1310 that links the binding domain of the binding-adaptor component 1312 to the binding domain 1308 is a derivative of succinimidyl 4[N-maleimidomethyl]cyclohexane-1-carboxylate (“SMCC”) 1314 that is bound to the thiolated 5′ end of the binding-domain oligonucleotide 1308 and bound to the binding-component 1312 through an amide bond. The binding component 1312 is a molecule of streptavidin. Streptavidin is a 52,800-dalton tetrameric protein purified from the bacterium Streptomyces avidinii. Streptavidin binds with an extremely strong affinity to biotin, with the dissociation constant of the biotin-streptavidin complex on the order of 10⁻¹⁵ mol/L. The binding domain of the target 1316 is a molecule of biotin 1318 that is covalently bound, through an amide linkage, to a target protein 1318.

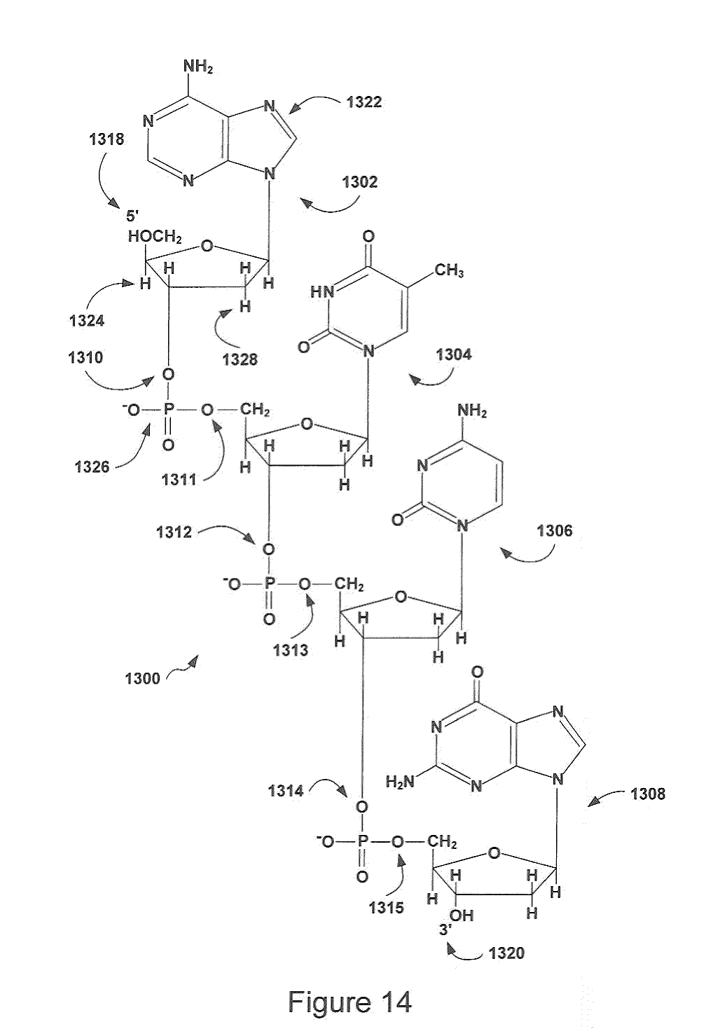

FIG. 14 illustrates a short oligonucleotide. The oligonucleotide is composed of the following subunits: (1) deoxy-adenosine 102; (2) deoxy-thymidine 104; (3) deoxy-cytosine 106; and (4) deoxy-guanosine 108. When phosphorylated, subunits of deoxyribonucleic-acid (“DNA”) and ribonucleic-acid (“RNA”) polymers are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1, can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilatc subunits of the other strand.

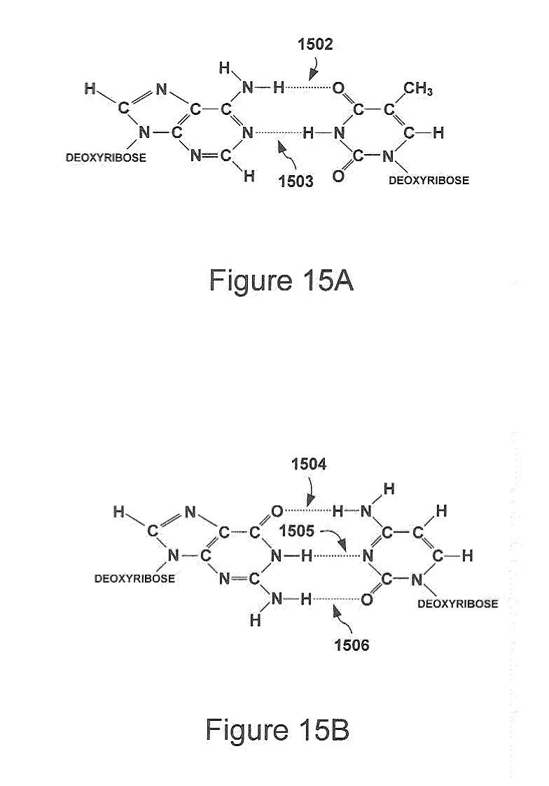

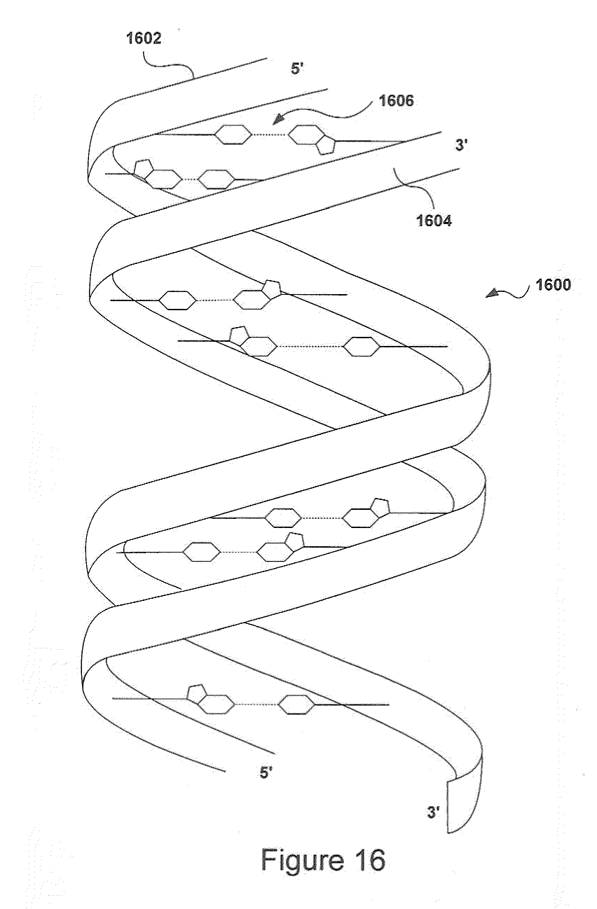

FIGS. 15A-B illustrate the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 16 illustrates a short section of a DNA double helix comprising a first strand and a second, anti-parallel strand.

The complementary binding of anti-parallel single-stranded oligonucleotides to produce oligonucleotide complexes through specific Watson-Crick interactions provides the ability to position the binding-adaptor component binding site anywhere along the binding-structure component of an oligonucleotide-based nanoscale tether that represents one embodiment of the present invention. In essence, the location of the binding-adaptor binding site is specified by the oligonucleotide sequence of the binding-adaptor binding domain. Thus, by choosing a binding-structure oligonucleotide without repeating subsequences of lengths similar to the lengths of the binding domains of the binding-adapter components, a binding-adaptor-component-binding-domain oligonucleotide sequence can be selected to bind at any position along the length of the oligonucleotide binding-structure component.

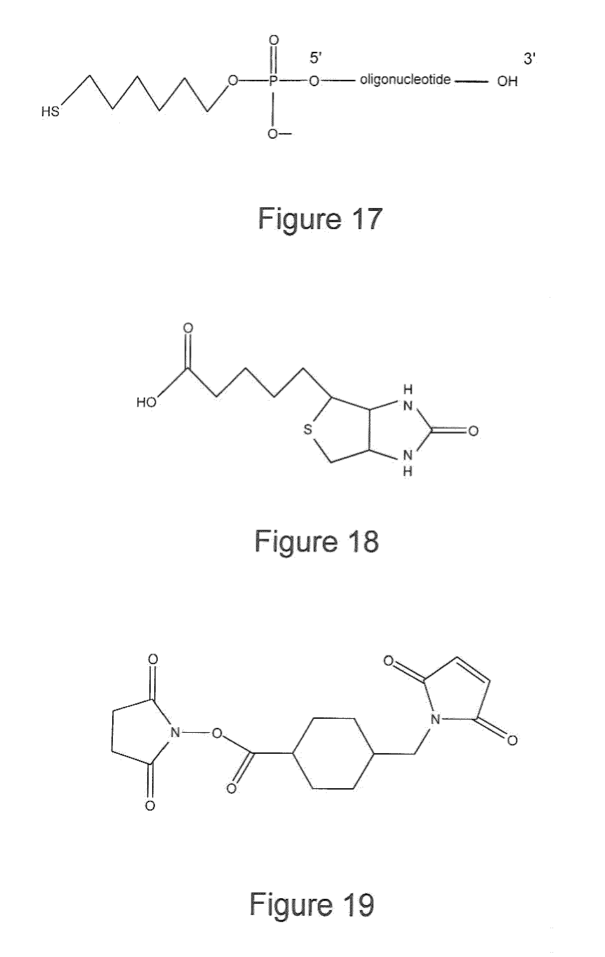

Finally, structural formulas are provided for particular nanoscale-tether subcomponents of one embodiment of the present invention. FIG. 17 shows the structural formula for a 5′-monothiolated. oligonucleotide. FIG. 18 shows the structural formula for biotin. FIG. 19 shows the structural formula for succinimidyl 4-[N-maleimidomethyl]cyclohexane-1-carboxylate (“SMCC”).

### EXPERIMENTAL RESULTS

### Example 1

### Transducer Surface Modification With Oligonucleotide

Four uL of 50 mM of dithiothreitol (DTT) solution in water was added to 100 uL of a solution of the 5′-thiolated oligonucleotide GGT AGT GCG AAA TGC CAT TGC TAG TTG TTT (10 uM in phosphate buffer saline) and incubated for one hour at ambient temperature. The solution was then applied to P6 micro-spin column to remove excessive DTT. The procedure was repeated with a fresh column. The obtained oligonucleotide solution (6 uL) was then applied to a cleaned gold surface of an interdigitated electrode and incubated at ambient temperature until it dried. The electrode was then washed multiple times with DI water.

### Example 2

### Oligonucleotide to Streptavidin Linkage via Primary Amines

### Thiol Modification of Oligonucleotidenucleotide

5′-thiolated oligonucleotide AAA CAA CTA GCA ATG GCA TTT (4 ul of 1 mM stock solution) was mixed with 90 ul of PBS and 10 ul of DTT (50 mM in water). The mixture was then incubated at ambient temperature for 4 hours and applied twice to P6 columns.

### Crosslinker Addition to Streptavidin

Succinimidyl 4-[N-maleimidomethyl]cyclohexane-1-carboxylate (SMCC) was dissolved in DMF to obtain a 1 mg/ml concentration. The SMCC solution (2.4 ul) was added to 100 ul of the streptavidin solution (1 mg/ml) in 1 × PBS. The mixture was then incubated for 2 hours at ambient temperature and applied twice to P6 columns.

### Conjugation Reaction

Modified streptavidin solution were combined with modified oligonucleotide and incubated for 2 hours at ambient temperature. The oligonucleotide linked immunoglobulin was then applied to P30 column to remove unbound oligonucleotide.

To obtain 3′ linked streptavidin oligonucleotide conjugate, the same technique was applied to a 3′-thiolated oligonucleotide.

### Example 3

### Biotinilation of Transglutaminase (TGA)

Transglutaminase was biotinylated using Pierce EZ Link Sulfo-NHS-LC-Biotinit kit according to Pierce protocol. Initial concentration of TGA was 1 mg/mL

### Example 4

### Impedance Spectroscopy Test: Anti-TGA Antibody Detection

Gamry instrument Reference 600 was used to perform an impedance spectroscopy test. The impedance spectroscopy test was performed at 150 mV and 26 Hz. The cell initially contained PBS with 0.05% Tween 20 (PBST) alone. After baseline drift was stabilized, PBST was removed from the cell and the sample was added. The addition of a sample containing 100 ng/mL anti TGA antibodies resulted in increase of impedance at 26 Hz scan frequency. When the transducer functionalized by streptavidin linked to the 3′ end of the oligonucleotide tag, the TGA is closer to the transducer surface than when the interdigitated electrode was functionalized by binding TGA-biotin to 5′ linked streptavidin oligonucleotide conjugate. Data analysis revealed that, for the 3′-end-linked-streptavidin conjugate, the rate of impedance increase is about 19% greater than for the 5′-end-linked streptavidin conjugate.

Although the present invention has been described in terms of particular embodiments, it is not intended that the invention be limited to these embodiments. Modifications will be apparent to those skilled in the art. For example, any of numerous polymers, macromolecular complexes, or nanoscale structures can be used for the binding-structure and binding-adaptor components of nanoscale tethers that represent embodiments of the present invention. As one example, an oligonucleotide binding-structure component may provide various different binding domains to a zinc-finger-binding-protein-based binding-adaptor component. Various synthetic oligonucleotide analogs may be used, in place of oligonucleotides, including oligonucleotides in which monomers are linked through sulfur-containing groups rather than through phosphodiester groups. Binding components of binding-adaptor components may include a wide variety of different target-binding-domain recognizing entities, including a wide variety of different proteins, nucleic acids, and other polymers that recognize and specifically bind to complementary molecules. As discussed above, the bound-target-to-substrate distance δₛ can be precisely controlled by positioning or defining the binding-domain-to-substrate distance δ_{y} of the binding-structure-component binding-adaptor binding domain. A variety of different substrate-surface preparation techniques and binding-component-fabrication techniques can be used to position binding structures at no more than a minimum distance from one another across the substrate of the sensor, in order to control bound-target-to-bound-target distances in addition to bound-target-to-substrate distances, to optimize and maximize signal-to-noise ratios, signal strength, and linearity of sensor-response curves.

1. A length-adjustable nanoscale tether comprising:
a binding-adapter component that includes a target-binding subcomponent to which a target binds, under experimental conditions, and a binding domain; and
a binding-structure component that includes a structural member, a substrate-mounting subcomponent that mounts the binding-structure component to a sensor substrate, and a binding-adapter binding domain to which the binding domain of the binding-adapter component binds, the binding-adapter binding domain positionable at different positions along the structural member with different, discrete binding-adapter-binding-domain-to-substrate distances δ_{y} corresponding to different, discrete bound-target-to-substrate distances δₛ that range from a minimum δₛ to a maximum δₛ.

2. The length-adjustable nanoscale tether of claim I wherein the binding-structure component has a polarity with respect to a longest dimension and wherein the binding-adapter component has a polarity with respect to the longest dimension of the binding domain of the binding-adapter component.

3. The length-adjustable nanoscale tether of claim 2 wherein the polarity of the binding-structure component is opposite from the polarity of the binding-adapter component when the binding-adapter component is bound to the binding-structure component.

4. The length-adjustable nanoscale tether of claim 2 wherein the binding-structure component has the same polarity as the binding-adapter component when the binding-adapter component is bound to the binding-structure component.

5. The length-adjustable nanoscale tether of claim 1 wherein each discrete bound-target-to-substrate distances δₛ represents a narrow range of distances due to flexibility in binding-adapter component.

6. The length-adjustable nanoscale tether of claim 1 wherein each discrete bound-target-to-substrate distances δₛ represents a narrow range of distances due to flexibility in the binding-adapter component.

7. The length-adjustable nanoscale tether of claim 1 wherein a binding-adapter component binds to a single binding-structure-component binding domain at a single distance δ_{y} from the sensor substrate.

8. The length-adjustable nanoscale tether of claim 1 wherein the discrete bound-target-to-substrate distances δₛ occur at regular, sub-nanoscale intervals within the range of minimum δₛ to maximum δₛ.

9. The length-adjustable nanoscale tether of claim 1 wherein the distance maximum δₛ-minimum δₛ is greater than 1 nanometer and less than 1 micrometer.

10. The length-adjustable nanoscale tether of claim 1 wherein the distance maximum δₛ-minimum δₛ is greater than 2 nanometers and less than 100 nanometers.

11. The length-adjustable nanoscale tether of claim 1 wherein the distance maximum δₛ-minimum δₛ is greater than 5 nanometers and less than 100 nanometers.

12. The length-adjustable nanoscale tether of claim 1 wherein the binding-structure component and the binding domain of the binding-adapter component both comprise a sequence of sub-nanoscale segments, the length of each segment equal to the difference between successive δₛ distances in the range from the minimum δₛ to the maximum δₛ.

13. The length-adjustable nanoscale tether of claim 1 wherein the binding-adapter component reversibly binds to the binding-structure component, so that the bind-adapter component or a binding-adapter/target complex can be removed from the binding-structure component.

14. The length-adjustable nanoscale tether of claim 1 wherein the target reversibly binds to the binding domain of the binding-adapter component, so that the target can be removed from the binding-structure component binding domain of the binding-adapter component.

15. The length-adjustable nanoscale tether of claim 1 further including a linker that links the target-binding subcomponent of the binding-adapter component to the binding domain of the binding-adapter component.

16. The length-adjustable nanoscale tether of claim 15 wherein the linker is a derivative of succinimidyl 4[N-maleimidomethyl]cyclohexane-1-carboxylate.

17. The length-adjustable nanoscale tether of claim 1 wherein the binding-structure component comprises an oligonucleotide.

18. The length-adjustable nanoscale tether of claim 17 wherein the binding-adapter binding domain is an oligonucleotide complementary to a subsequence of the binding-structure component.

19. The length-adjustable nanoscale tether of claim 1 wherein the binding subcomponent of the binding-adapter-component binding subcomponent is streptavidin.

20. The length-adjustable nanoscale tether of claim 17 wherein the target includes a biotin molecule that binds to the streptavidin or avidin binding-adapter-component binding subcomponent.

21. The length-adjustable nanoscale tether of claim 17 wherein the substrate-mounting subcomponent is a thiolated long-chain alcohol molecule.

22. A method for preparing a sensor for detecting a target, the method comprising:
preparing a sensor substrate to receive binding-structure components of length-adjustable nanoscale tethers; and
binding binding-structure components of length-adjustable nanoscale tethers to the prepare substrate.

23. The method of claim 22 further including binding, to the binding-structure components of the length-adjustable nanoscale tethers, binding-adapter components that bind to the binding-structure components at a distance δ_{y} from the sensor substrate.

24. The method of claim 22 further including binding, to the binding-structure components of the length-adjustable nanoscale tethers, binding-adapter-component/target complexes that bind to the binding-structure components at a distance δ_{y} from the sensor substrate.
